(19) United States
(12) Patent Application Publication
(10) Pub. No.: US 2017/0164843 A1
(43) Pub. Date: Jun. 15, 2017

(54) BLOOD PRESSURE SENSOR

(71) Applicant: Infineon Technologies AG, Neubiberg (DE)

(72) Inventors: Hans Ehm, Pullach (DE); Dirk Hammerschmidt, Finkenstein (AT); Kristina Vaupel, Muenchen (DE)

(21) Appl. No.: 15/308,967

(22) PCT Filed: May 7, 2015
(86) PCT No.: PCT/EP2015/000928

§ 371 Date: Dec. 28, 2016

(30) Foreign Application Priority Data
May 8, 2014 (DE) 10 2014 006 726.2

(51) Int. Cl. A61B 5/0215; A61B 5/00; A61B 5/0205

(52) U.S. Cl. CPC A61B 5/0215; A61B 5/0205; A61B 5/0031; A61B 5/6876; A61B 5/0024; A61B 2560/0209; A61B 2560/0219; A61B 2503/40; A61B 2503/42

(57) ABSTRACT

The disclosure relates to a system for monitoring at least one physiological parameter. The system comprises a measuring apparatus having a sensor unit for detecting the at least one physiological parameter and having a transmission device, and a reading apparatus for receiving data relating to the at least one physiological parameter. The transmission device is activated when the measuring apparatus lies within the range of the reading apparatus.

The present invention relates to sensor apparatuses and to a method and a system for monitoring physiological parameters. In particular, it relates to the monitoring of the blood pressure of laboratory animals.

For many medical, diagnostic and therapeutic measures, it is expedient, desirable or necessary to determine and possibly monitor the blood pressure under the physiological parameters. In order to measure and monitor blood pressure and other parameters, automated systems which allow maximally continuous measurement and monitoring are desirable.

### SUMMARY OF THE INVENTION

The present description provides a system, a method and an apparatus for measuring at least one vital parameter, for example the blood pressure, as claimed in one of the independent claims. Further examples and features are specified in the dependent claims.

The apparatus comprises a sensor device for detecting the at least one physiological parameter, a memory device for electronic storage of data relating to the at least one physiological parameter, a transmission device for wireless transmission of the data relating to the at least one physiological parameter, and an energy storage unit which supplies at least one of the sensor devices and the memory device with electricity. The transmission device can be coupled inductively to an external electricity supply.

### BRIEF DESCRIPTION OF THE FIGURES

FIG. 1 shows the way in which a measuring apparatus of the present description may be arranged on a mouse;

FIG. 2 shows the positions at which a blood pressure sensor may advantageously be arranged on a heart or a blood vessel near the heart;

FIG. 3 shows a measuring apparatus in more detail;

FIG. 4 shows a block diagram of a measuring apparatus; and

FIGS. 5a and b show a system for use of the measuring apparatus in a cage.

### DETAILED DESCRIPTION

Exemplary embodiments of the invention will be explained in more detail below with reference to the appended figures. The invention is not, however, restricted to the embodiments specifically described, but may be modified and altered in a suitable way. It is within the scope of the invention to combine individual features and feature combinations of one embodiment suitably with features and feature combinations of another embodiment, in order to obtain further embodiments according to the invention.

Before the exemplary embodiments of the present invention are explained in more detail below with the aid of the figures, it is to be pointed out that elements which are the same in the figures are provided with the same or similar references, and that repeated description of these elements is omitted. Furthermore, the figures are not necessarily true to scale. Rather, the focus is on explaining the basic principle.

In one aspect, the present description discloses a measuring apparatus for measuring or determining a physiological parameter. The physiological parameter may be the blood pressure. In addition or as an alternative, other physiological parameters may be measured, for example body temperature, heart rate, blood sugar, volume variation, pH values and other physiological parameters. To this end, the measuring apparatus comprises a sensor device having e.g. a pressure sensor, which may be implanted and arranged in or on a blood vessel. For example, the sensor device may be arranged internally or externally on a vessel wall, or alternatively in the heart.

In another aspect, the description relates to a system for determining the at least one physiological parameter.

In the description below, the measuring apparatus, the system and the method will be explained in more detail with reference to the example of mice which are kept in a cage. This application may be used for monitoring laboratory mice or other laboratory animals.

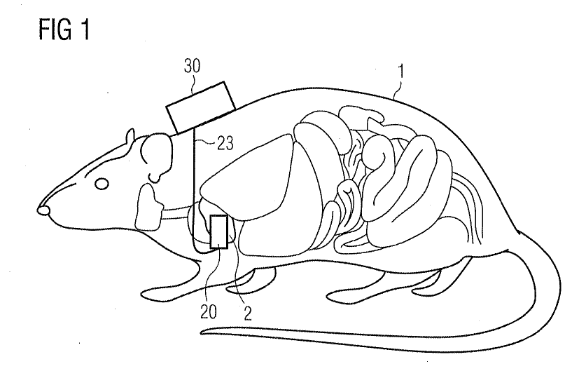

FIG. 1 shows an example of the way in which a measuring apparatus of the present description may be used. The measuring apparatus comprises a carrier unit 30. The carrier unit may for example comprise a housing, a carrier film, a PCB (printed circuit board) or the like, which is fastened on the back of a mouse 1, preferably below the skin, i.e. subcutaneously, and is carried by this mouse without hindrance or restriction in its freedom of movement. As an alternative to implantation under the skin, the carrier or the housing could also be fastened on the mouse in the form of a backpack or a body belt. Various electronic components are arranged in the housing, or on the carrier, as described below. A sensor device 20 is connected to the carrier device 30 by an electrical cable 23. The electrical cable 23 may be a flexible microcable (microwire), and may likewise consist of the material of the carrier film. As an alternative to a cable, a wireless connection is also possible.

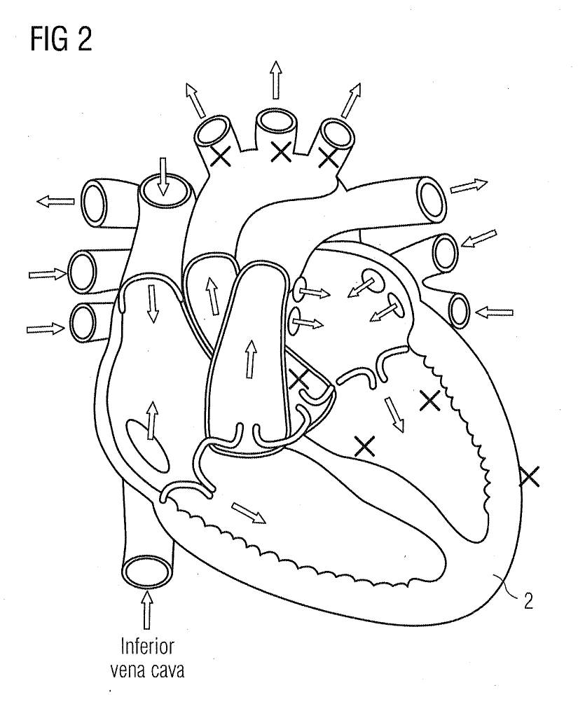

The sensor device 20 of FIG. 1 comprises one or more blood pressure sensors and is implanted in a blood vessel of the mouse 1. FIG. 2 shows a heart 2, in which the positions at which the sensor device 20 may′ particularly favorably be arranged or implanted are marked by crosses. For example, the sensor device 20 may be arranged in the left heart chamber, which is the aorta, in order to allow particularly reliable and accurate determination of the blood pressure. The specific position may also be selected by the person carrying out the implantation according to the situation and requirements, for example in the carotid artery. It is also possible to arrange a plurality of blood pressure sensors, or even sensor devices different to one another, at different positions in or on the heart 2 in different vessels. In this way, it is possible not only to determine the blood pressure or another physiological parameter at a single position, but also to determine differences or distributions, for example blood pressure differences or distributions at one or more positions, independently, in groups or in combination.

For example, one blood pressure sensor may be arranged on the aorta and another blood pressure sensor may be arranged on the vena cava and/or at different positions of the heart.

Each of the sensor devices 20, or each blood pressure sensor, may be connected to the carrier device 30 by means of a separate flexible microcable 23.

The fastening, or anchoring, of the sensor device 20 on the blood vessel may for example be carried out by means of a glue or another adhesive, these being known per se and available. It is also possible to anchor or suture the sensor device 20 in tissue pockets. It is possible to combine a variety of these fastening possibilities with one another. It is also possible to suspend a plurality of sensors, or a sensor unit, in the bloodstream, for example by means of microcable anchored on the vessel wall.

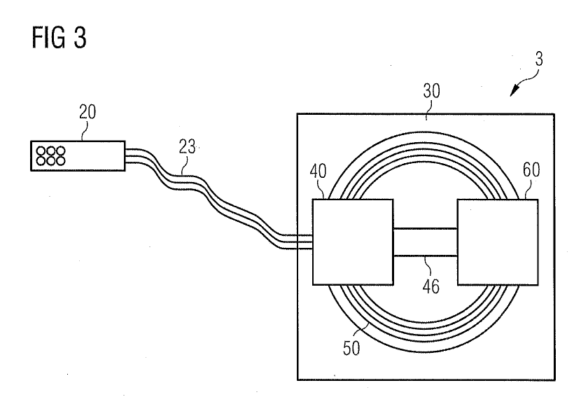

FIG. 3 schematically shows a measuring apparatus 3 of one example of the present description. The sensor device 20, as also represented in FIG. 1, is connected to the carrier device 30 by means of the flexible microcable 23. Arranged in the carrier device 30, there is a transmission device having a transponder and a resonator in the form of a coil 50 as an antenna. The transponder 40 may be configured for NFC (near-field communication) for another wireless transmission technology.

The coil 50 may, for example, be printed on a flexible film. Arranged in or on the carrier device 30, there is furthermore an energy storage unit 60, which is connected to the transponder and optionally the sensor unit 20 by means of an electrical connection 46 and supplies them with electrical energy. The energy storage unit 60 may be in the form of a battery, and may be configured as a primary or secondary battery. A secondary battery may, for example, be charged by a current induced in the coil 50. Preferably, a battery in flat design is used, for example a silicon battery with a thickness similar to ICs. As an alternative, however, by accepting a larger design, it is also possible to use a conventional button battery. As an alternative, a thin-film battery may also be used. The battery 60 may also be integrated in the film of the coil 50.

Besides a housing as the carrier device 30, it is also possible to fasten the transponder 40 and the battery 60, arranged on the coil 50, and provided for example with a protective layer, directly on the mouse 1 or implant them under its skin.

The sensor unit 20 may be dimensioned in terms of spatial requirement in such a way as not to hinder or interfere with its arrangement on the blood vessel of the animal. All elements with a higher spatial requirement may be fitted in the carrier unit. This relates above all to the transponder 40, and in many cases the battery 60. In comparison with a catheter, the hindrance to the animal and the impairment of its freedom of movement are therefore reduced significantly.

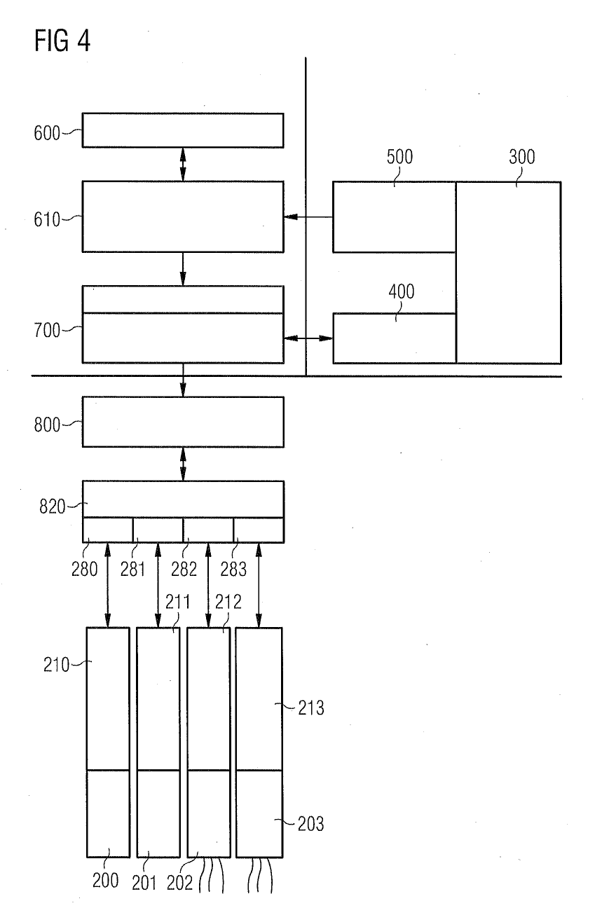

FIG. 4 shows a block diagram of the measuring apparatus 3. In the example represented here, the measuring apparatus comprises a sensor device having four sensors 200, 201, 202, 203. Besides the aforementioned blood pressure sensor 200, a temperature sensor 201, an ECG (electrocardiograph) probe 202 and an electrical stimulator 203 may for example also be provided here, these being referred to as sensors 200, 201, 202, 203 for brevity. The sensors are merely examples, and other sensors may be used depending on the situation. Nonexhaustive examples comprise pH sensors, ion-specific sensors, blood sugar sensors, extension or volume sensors, resistance measuring apparatuses or other biosensors.

It is also possible to connect a plurality of blood pressure sensors in order to determine the blood pressure at different positions, or a combination of blood pressure and other sensors. For example, a network or array of sensors may be used in order to take measurement values at different positions. An array of sensors arranged on the heart may, for example, detect failures or dysfunctions of particular regions of the heart, and therefore detect myocardial infarctions or voltage peaks during fibrillations. It is also possible to apply an array of sensors to different positions of the body, and thus obtain information about the blood pressure distribution. In this case, the sensors may be all blood pressure sensors or a combination of different sensors.

Besides sensors, it is also possible to arrange actuators such as medicament delivery devices, stimulators or defibrillators individually or in arrays.

These may be driven in a similar way to the sensors or be means of a separate controller.

The number four of sensors indicated here is therefore merely exemplary, and any other number of sensors may be provided. The sensors 200, 201, 202, 203 may be arranged at different positions on the mouse. The temperature sensor 201 may, for example, be combined with the blood pressure sensor 200 in a unit or on a semiconductor chip, and measure the temperature directly in the blood vessel. The temperature sensor 201 may, however, also be arranged at a different position, for example on the transponder 40. Furthermore, the electronics of the sensors may also be partially or fully integrated in the same chip as the transponder 40.

In the sensor device, each of the sensors 200, 201, 202, 203 is assigned a data preparation unit or sensor controller 210, 211, 212, 213, which controls the respective sensor and optionally prepares the signals recorded by the sensor for further processing.

The sensors 200, 201, 202, 203 and the data preparation units or sensor controllers 210, 211, 212, 213 are connected by means of respective connections 280, 281, 282, 283 to a sample interface 820. The sample interface 820 may be part of the sensor device 20, and is connected to a processor 800. As an alternative, the sample interface 820 may also be integrated into the processor 800 which controls the measurements and optionally prepares, for example compresses, the measurement data. Furthermore, the processor 800 is connected to a data memory 700 which can store the data relating to the measurements. Configurations may also be stored here. The data memory 700 may be a RAM or flash memory.

As an alternative or in addition, the sensors 200, 201, 202, 203 and the data preparation units or sensor controllers 210, 211, 212, 213 may also be constructed as a grid, array or network, in which case the sensors 200, 201, 202, 203 may, for example, be arranged at different positions in and/or on the body. Each data preparation unit or sensor controller 210, 211, 212, 213 may in this case have an interface and communicate directly via the processor 800 with sensor controllers 210, 211, 212, 213 of other sensors. In this way, for example, after measurement of a particular value, a first sensor may request measurement at other positions or measurement of other parameters, or even activate actuators.

The grid or network may consist of sensors of the same type, for example blood pressure sensors may be arranged at different positions of an organ (for example the heart) for the entire body. It is, however, also possible to combine any desired combinations of sensors and/or actuators in a grid/network in any desired way and according to requirements.

The processor 800, the sample interface 820, the sensors 200, 201, 202, 203 and the sensor controllers 210, 211, 212, 213, and optionally the data memory 700, are supplied with electricity by the battery 600 constantly or when required. This ensures that the measurements and the processing and storage of the measured parameter values can be carried out at any time, continuously and independently of the position of the mouse.

In addition, a system monitor 610 may be provided, for example having a voltage regulator, energy and/or battery management, a wake-up timer or a time controller, and a control processor. With the wake-up timer, or the time controller, measurements may for example be carried out at predetermined intervals, the system monitor activating the sensors, the processor and the data memory at the predetermined times or intervals' with the wake-up timer and initiating the measurement, and remaining active until the measured data have been stored in the memory 700. The energy management or the time controller is constantly supplied with energy. All other units are switched on or off according to requirements and the operating state. Possible operating states comprise, for example autonomous, read, charge, measure, configure. A requirement may for example represent measurement, compression, storage, sleep, but may also involve other processes of the apparatus.

Besides this battery-operated area, a data transmission device having the transponder 400 and an antenna 500 is provided. The transponder 400 and the antenna 500 are not supplied with electricity by the battery 600, or are supplied only for particular restricted operating states. By means of the antenna 500, which may for example be configured as a near-field communication (NFC) device, for example in the form of an LC resonator, the transponder 400 may be supplied with electricity when the antenna 500 lies within the range of a near-field reading device 90. A voltage, which wakes up the transponder and starts data transmission, may be inductively coupled in via antenna 500.

The transponder therefore remains passive so long as it is not being supplied with current, or a voltage, via the antenna 500, and is only activated by the voltage coupled in. This has the advantage that the battery is not consumed for the data transmission to the reader, but is used only for the less energy-consuming measurement and storage of the data. At the same time, by means of the battery the measurements may be carried out independently of the position of mouse, and the lifetime of the battery and therefore the total measurement time can be extended substantially and/or the battery can be made correspondingly smaller and therefore lighter in order to restrict the animal less.

The transponder may also be used in order to receive configuration data from the near-field reader 90 and configure picture measurements accordingly. That is to say, the near-field reader 90 may be configured not only in order to read the data act, but, as a transceiver, also for transmitting data. For example, the energy management for the wake-up function may be reconfigured and other measurement intervals or instants or other control sequences may be programmed and set.

It is also possible to reconfigure one or more parameters for the sensors and/or the measurements. Nonexhaustive examples of parameters which may be reconfigured comprise sleep duration, sampling rate, number of measurement points per waking period, compression rate and compression method. These and other parameters may be configured individually for each sensor.

Lastly, the voltage coupled in inductively/the current coupled in inductively, may also be used in order to charge the battery 600, when it is a rechargeable battery.

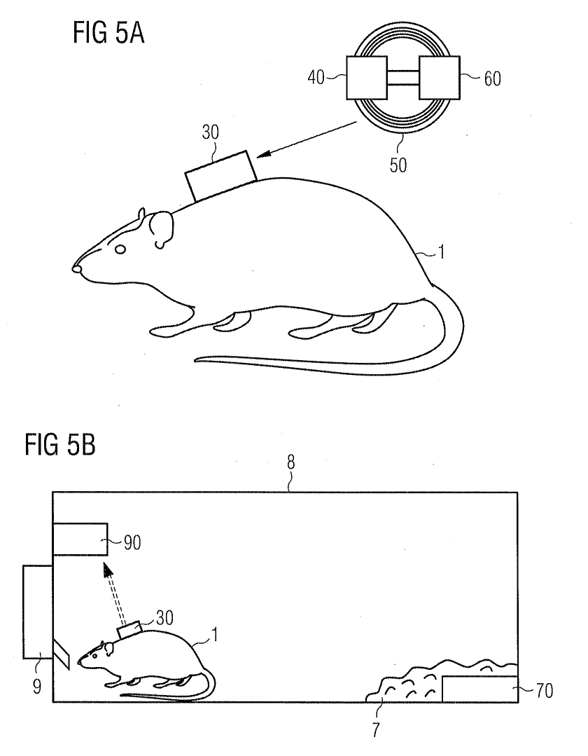

FIG. 5a shows the arrangement of the carrier device 30 with the transponder 40, antenna 50 and battery 60 on the mouse 1. FIG. 5b shows the mouse 1 with the carrier device 30 of the measuring apparatus in a cage 8, in which case the carrier device 30 may also be housed, packaged or configured as a housing. The cage may be a laboratory cage, such as is conventional for holding laboratory mice.

A feeding point and/or watering point 9, at which the mouse 1 can respectively drink and eat, is arranged on the cage 8, as is conventional for laboratory cages. The near-field reader 90 is arranged in the vicinity of the feeding point and/or watering point 9. When the mouse with the measuring apparatus, and therefore with the antenna 50, comes to eat or drink and enters the range of the near-field reader 90, data transmission to and from the transponder 40 is activated by means of the near-field reader 90, and the measured data and store data are transmitted. At the same time, if desired, the measuring apparatus may be reconfigured or programmed, bidirectional exchange of data between the near-field reader 90 and the responder being possible.

The near-field reader 90 supplies the transponder key inductively with electricity, and is therefore used as the electricity supply for the data transmission, the battery 60, 600 may be charged.

A typical time for which the mouse 1 remains at the feeding point and/or watering point 9 is readily sufficient for the data transmission. However, this time may be too short for efficient charging of the battery. A separate charging apparatus 70 may therefore be provided for the charging. This may for example be provided in the vicinity of the sleeping location, so that the charging process may take place during sleep. It is furthermore possible to fit the charging apparatus 70 on one of the inner cage walls or on the upper cage roof. Furthermore, the charging apparatus may extend into the cage or be suspended from the roof. It is also possible to fit the charging apparatus 70 outside the cage within the range of the near-field reader 90. For example, the charging apparatus 70 may be fitted to or on the outer cage walls.

Substantially longer charging times are therefore possible, and the maximum number of measurements can be increased extensively, so that, depending on the configuration of the system, the measurement duration may be extended or the sampling rate may be increased.

The entire system has no effect on the mouse, since cable connections are not required. The mouse can move without interference.

It is also possible to assign each measuring apparatus 3, or even each sensor unit 20, with unique identification, for example in the form of an ID code, which can be transmitted together during the data transmission. This ID code may already be written into a chip during manufacture. As an alternative or in addition, a memory element which can be written to once or several times may also be provided, in which an identification code and optionally further data about the animal can be stored.

This allows unique identification and assignment of individual animals, for example in group housing.

This also allows trackability and better checking of the measurement data. In addition or as an alternative, the identification code may also be applied in an externally readable way on a housing of the carrier device. This way, the animal can be identified immediately at a glance.

The description has been given by way of example with reference to animals which are kept in cages. Individual aspects or several aspects may, however, also be applied in other situations and with other animals. For example, similar systems and apparatuses may be envisioned with animals which are kept in a shed or zoo. In the case of animals attracted by food, use with animals living wild is also possible.

In principle, use with humans is also conceivable. In this case, the readout may occur at places which are actively sought, by fitting the transmission unit in the vicinity of the reading apparatus.

1. An apparatus for determining at least one physiological parameter, the apparatus comprising:
a sensor device for detecting the at least one physiological parameter;
a memory device for electronic storage of data relating to the at least one physiological parameter; and
a transmission device for wireless transmission of the data relating to the at least one physiological parameter,
an energy storage unit which supplies at least one of the sensor devices and the memory device with electricity,
wherein the transmission device can be coupled inductively to an external electricity supply.

2. The apparatus as claimed in claim 1, wherein the sensor device comprises at least one blood pressure sensor, and the at least one physiological parameter is blood pressure.

3. The apparatus as claimed in claim 1, wherein at least the sensor device is configured for implantation in a blood vessel.

4. The apparatus as claimed in claim 1, wherein the transmission device comprises a transponder and a resonator.

5. The apparatus as claimed in claim 1, wherein the transmission device and the battery are fitted on a carrier unit, which is connected to the sensor unit by means of a cable.

6. The apparatus as claimed in claim 1, furthermore comprising a processor, which processes the measurement data of the sensor device and provides the data relating to the at least one physiological parameter.

7. The apparatus as claimed in claim 6, wherein the processor controls the sensor device.

8. A system for monitoring at least one physiological parameter, the sensor comprising:
a measuring apparatus having a sensor unit for detecting the at least one physiological parameter and having a transmission device;
a reading apparatus for receiving data relating to the at least one physiological parameter,
wherein the transmission device is activated when the measuring apparatus lies within the range of the reading apparatus.

9. The system as claimed in claim 8, wherein the measuring apparatus comprises an energy storage unit and a data memory, the energy storage unit supplying at least one data memory and the sensor unit with electricity.

10. The system as claimed in claim 8, wherein the reading apparatus inductively supplies at least the transmission device with electricity.

11. The system as claimed in claim 8, wherein the sensor device comprises a blood pressure sensor.

12. The system as claimed in claim 8, wherein the sensor device is configured for implantation.

13. The system as claimed in claim 8, furthermore comprising a cage, wherein the reading apparatus is arranged on the cage.

14. The system as claimed in claim 13, further comprising an active charging station, which is arranged on the cage.

15. The system as claimed in claim 8, furthermore comprising an energy management unit, which contains a time controller that is constantly supplied with energy and switches all the other units on or off according to requirements and the operating state.

16. The system as claimed in claim 15, wherein a control sequence of the energy management unit can be reconfigured by means of near-field communication.

17. The system as claimed in claim 15, wherein at least one of the parameters: sleep duration, sampling rate, number of measurement points per waking period, compression rate, compression method, for at least one of the sensors can be programmed by means of near-field communication.
